(19) United States
(12) Patent Application Publication
(10) Pub. No.: US 2026/0294750 A1
(43) Pub. Date: Oct. 1, 2026

(54) FOAMING CLEANSER WITH FATTY ALCOHOL THICKENERS

(71) Applicant: L'OREAL, Paris (FR)

(72) Inventors: Changlong Chen, Edison, NJ (US); Christopher Pang, New York, NY (US); Hungta Lin, Teaneck, NJ (US); Mariko Hasebe, New York, NY (US)

(73) Assignee: L'OREAL, Paris (FR)

(21) Appl. No.: 19/093,788

(22) Filed: Mar. 28, 2025

(51) Int. Cl. A61K 8/04; A61K 8/34; A61K 8/44; A61Q 19/00; A61Q 19/10

(52) U.S. Cl. CPC A61K 8/046; A61K 8/342; A61K 8/447; A61Q 19/005; A61Q 19/10; A61K 2800/30; A61K 2800/596; A61K 2800/72

(57) ABSTRACT

A foaming cleanser is disclosed, comprising at least one fatty alcohol, at least one amphoteric surfactant, at least one anionic surfactant, and water, wherein the formulation is free of PEG based ingredients. In some embodiments, the cleanser formulation is further characterized by the exclusion of PEG/PEO polymeric thickeners, parabens, and combinations thereof. The cleanser may specifically exclude PEG-150 pentaerythrityl tetrastearate, PEG-6 caprylic/capric glycerides, or combinations thereof, providing a milder and more environmentally friendly alternative for skin care applications. This formulation ensures effective cleansing and foaming properties while minimizing potential irritants, making it suitable for all skin types, including sensitive skin.

### FIELD

The present invention relates to a foaming cleanser that comprises at least one fatty alcohol, at least one amphoteric surfactant, at least one anionic surfactant, and water, wherein the foaming cleanser excludes PEG based ingredients.

### BACKGROUND

Foaming cleansers have been widely used in personal care products for their ability to effectively cleanse the skin while providing a luxurious foaming experience. Traditional formulations of foaming cleansers often include polyethylene glycol (PEG) based ingredients as thickeners or stabilizers, which help in achieving the desired consistency and performance characteristics. However, the use of PEG based ingredients has raised concerns due to potential skin irritation and environmental impact. Accordingly, these ingredients, while effective, are not always desirable due to their synthetic nature and potential to cause skin sensitivity in some users.

The problem addressed by the present invention is to provide a foaming cleanser formulation that effectively cleanses and foams without the inclusion of PEG based ingredients, while maintaining the desired aesthetic and functional properties. This invention meets consumer demand for more natural and skin-friendly alternatives in personal care products, offering an improved formulation that aligns with current trends towards cleaner and more environmentally friendly beauty products.

### SUMMARY

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

According to an embodiment of the invention, a foaming cleanser comprises (a) at least one fatty alcohol; (b) at least one amphoteric surfactant; (c) at least one anionic surfactant; and (d) water; wherein the foaming cleanser excludes PEG based ingredients. This formulation provides a skin-friendly cleansing solution that avoids potential irritants associated with PEG based ingredients.

In some embodiments, the foaming cleanser excludes polyethylene glycol/polyethylene oxide (PEG/PEO) polymeric thickeners, parabens, and combinations thereof. This exclusion further enhances the safety profile of the cleanser for sensitive skin. In some embodiments, the foaming cleanser excludes PEG-150 pentaerythrityl tetrastearate, PEG-6 caprylic/capric glycerides, or a combination thereof, reducing the risk of adverse skin reactions. In some embodiments, the at least one fatty alcohol is present from about 0.05% to about 2% by weight, based on the weight of the composition. In some embodiments, the at least one fatty alcohol is present from about 0.05% to about 1% by weight, based on the weight of the composition, ensuring effective skin conditioning. In some embodiments, the fatty alcohol is present in a total amount that does not exceed 2% by weight, based on the weight of the composition. In some embodiments, the at least one fatty alcohol is characterized by a feature selected from the group consisting of (i) being liquid or semi-waxy at room temperature and containing carbon chain length from 6 up to 22; (ii) being liquid or semi-waxy at room temperature and containing carbon chain length from 6 up to 18; (iii) being liquid or semi-waxy at room temperature and containing carbon chain length from 8 up to 10; (iv) being liquid or semi-waxy at room temperature and containing carbon chain length from 10 up to 16; (v) being selected from linear or branched, saturated or unsaturated, with hydroxyl group on terminal position or non-terminal position; (vi) being selected from the group consisting of hexyldecanol, oleoyl alcohol, lauryl alcohol, myristyl alcohol, cetyl alcohol, decanol, butyloctanol, hexydecanol, palmitoleyl alcohol, and combinations thereof; and (vii) combinations thereof. In some embodiments, the at least one amphoteric surfactant is characterized by a feature selected from the group consisting of (i) present from about 1% to about 15% by weight, based on the weight of the composition; (ii) present from about 3% to about 10% by weight, based on the weight of the composition; (iii) selected from the group consisting of cocoamidopropyl hydroxysultaine, coco betaine, cocoamidopropyl betaine, and combinations thereof; and (iv) combinations thereof. In some embodiments, the at least one anionic surfactant comprises sodium methyl cocoyl taurate. In some embodiments, the foaming cleanser further comprises an additional ingredient selected from the group consisting of niacinamide, phenoxyethanol, sodium hyaluronate, glycerin, sodium chloride, sodium benzoate, sodium lauroyl lactylate (and) ceramide NP (and) ceramide AP (and) phytosphingosine (and) cholesterol (and) xanthan gum (and) carbomer (and) ceramide EOP, propanediol, tetrasodium glutamate diacetate, citric acid, and combinations thereof, which can enhance the overall skin benefits of the cleanser.

According to an embodiment of the invention, a foaming cleanser comprises (a) at least one fatty alcohol present from about 0.05% to about 2% and selected from the group consisting of decanol, butyloctanol, lauryl alcohol, myristyl alcohol, hexydecanol, palmitoleyl alcohol, oleoyl alcohol, and combinations thereof; (b) at least one amphoteric surfactant present from about 1% to about 15% and selected from the group consisting of cocoamidopropyl hydroxysultaine, coco betaine, cocoamidopropyl betaine, and combinations thereof; (c) at least one anionic surfactant present from about 1% to about 15% and selected from the group consisting of sarcosinate, glycinate, glutamate, taurate, isethionate, alpha olefin sulfonate, and combinations thereof; all amounts by weight, based on the weight of the composition.

In some embodiments, the foaming cleanser excludes PEG/PEO polymeric thickeners, parabens, and combinations thereof. In some embodiments, the at least one fatty alcohol consists of (i) hexyldecanol, (ii) oleoyl alcohol, or (iii) a blend of lauryl alcohol, myristyl alcohol, and cetyl alcohol, ensuring targeted skin benefits and formulation stability. In some embodiments, the at least one amphoteric surfactant is present from about 3% to about 10% by weight, based on the weight of the composition, and the at least one anionic surfactant is present from about 3% to about 10% by weight, based on the weight of the composition. In some embodiments, each of the at least one fatty alcohol is liquid at room temperature and contains a carbon chain length from 8 up to 10, and the at least one amphoteric surfactant contains a carbon chain length distribution from C8-10, or each of the at least one fatty alcohol is liquid at room temperature and contains a carbon chain length from 16 up to 18, and the at least one amphoteric surfactant contains a carbon chain length distribution from C16-18. In some embodiments, the foaming cleanser further comprises an additional ingredient selected from the group consisting of niacinamide, phenoxyethanol, water/aqua, sodium hyaluronate, glycerin, sodium chloride, sodium benzoate, sodium lauroyl lactylate (and) ceramide NP (and) ceramide AP (and) phytosphingosine (and) cholesterol (and) xanthan gum (and) carbomer (and) ceramide EOP, propanediol, tetrasodium glutamate diacetate, citric acid, and combinations thereof, enhancing the multifunctional benefits of the cleanser.

According to an embodiment of the invention, a foaming cleanser comprises (a) at least one fatty alcohol present from about 0.05% to about 2% and selected from the group consisting of (i) hexyldecanol, (ii) oleoyl alcohol, (iii) a blend of lauryl alcohol, myristyl alcohol, and cetyl alcohol, (iv) and combinations thereof, wherein the fatty alcohol is present in a total amount that does not exceed 2% by weight, based on the weight of the composition; (b) at least one amphoteric surfactant present from about 3% to about 10% and comprising at least cocoamidopropyl hydroxysultaine; (c) at least one anionic surfactant present from about 3% to about 10% and comprising at least sodium methyl cocoyl taurate; (d) an additional ingredient selected from the group consisting of niacinamide, phenoxyethanol, water/aqua, sodium hyaluronate, glycerin, sodium chloride, sodium benzoate, sodium lauroyl lactylate (and) ceramide NP (and) ceramide AP (and) phytosphingosine (and) cholesterol (and) xanthan gum (and) carbomer (and) ceramide EOP, propanediol, tetrasodium glutamate diacetate, citric acid, and combinations thereof; all amounts by weight, based on the weight of the composition; wherein the foaming cleanser excludes PEG/PEO polymeric thickeners, parabens, and combinations thereof.

These and other aspects of the invention are set out in the appended claims, and described in greater detail in the detailed description of the invention.

It is to be understood that both the foregoing general description and the following detailed description are exemplary and explanatory only and are not restrictive of the disclosure.

### DETAILED DESCRIPTION

In the following detailed description, the components of the foaming cleanser are outlined, adhering to the format of the guiding application. Each category of ingredients is discussed in terms of its function, typical concentration, and examples of suitable compounds.

### Foaming Cleanser

The disclosure provides, in various embodiments, a foaming cleanser comprises (a) at least one fatty alcohol; (b) at least one amphoteric surfactant; (c) at least one anionic surfactant; and (d) water; wherein the foaming cleanser excludes PEG based ingredients. This formulation provides a skin-friendly cleansing solution that avoids potential irritants associated with PEG based ingredients. In some embodiments, the foaming cleanser excludes PEG/PEO polymeric thickeners, parabens, and combinations thereof. This exclusion further enhances the safety profile of the cleanser, particularly for, but not limited to, sensitive skin. In some embodiments, the foaming cleanser excludes PEG-150 pentaerythrityl tetrastearate, PEG-6 caprylic/capric glycerides, or a combination thereof, reducing the risk of adverse skin reactions. In some embodiments, the at least one fatty alcohol is present from about 0.05% to about 2% by weight, based on the weight of the composition, providing optimal moisturizing properties without excessive greasiness.

In some embodiments, the at least one fatty alcohol is present from about 0.05% to about 1% by weight, based on the weight of the composition, ensuring effective skin conditioning. In some embodiments, the fatty alcohol is present in a total amount that does not exceed 2% by weight, based on the weight of the composition. In some embodiments, the at least one fatty alcohol is characterized by a feature selected from the group consisting of (i) being liquid or semi-waxy at room temperature and containing carbon chain length from 6 up to 22; (ii) being liquid or semi-waxy at room temperature and containing carbon chain length from 6 up to 18; (iii) being liquid or semi-waxy at room temperature and containing carbon chain length from 8 up to 10; (iv) being liquid or semi-waxy at room temperature and containing carbon chain length from 10 up to 16; (v) being selected from linear or branched, saturated or unsaturated, with hydroxyl group on terminal position or non-terminal position; (vi) being selected from the group consisting of hexyldecanol, oleoyl alcohol, lauryl alcohol, myristyl alcohol, cetyl alcohol, decanol, butyloctanol, hexydecanol, palmitoleyl alcohol, and combinations thereof; and (vii) combinations thereof, providing versatility in formulation to achieve desired sensory and functional attributes.

In some embodiments, the at least one amphoteric surfactant is characterized by a feature selected from the group consisting of (i) present from about 1% to about 15% by weight, based on the weight of the composition; (ii) present from about 3% to about 10% by weight, based on the weight of the composition; (iii) selected from the group consisting of cocoamidopropyl hydroxysultaine, coco betaine, cocoamidopropyl betaine, and combinations thereof; and (iv) combinations thereof, ensuring mild and effective cleansing action.

In some embodiments, the at least one anionic surfactant comprises sodium methyl cocoyl taurate.

In some embodiments, the foaming cleanser further comprises an additional ingredient selected from the group consisting of niacinamide, phenoxyethanol, sodium hyaluronate, glycerin, sodium chloride, sodium benzoate, sodium lauroyl lactylate (and) ceramide NP (and) ceramide AP (and) phytosphingosine (and) cholesterol (and) xanthan gum (and) carbomer (and) ceramide EOP, propanediol, tetrasodium glutamate diacetate, citric acid, and combinations thereof, which can enhance the overall skin benefits of the cleanser.

The foaming cleanser is shelf stable and stable on application, demonstrating a viscosity of about 15-40 UD (Viscosity Rheomat M3 Spindle), or from about 20-30 UD, and a pH about 4.5 to about 6.5, or about pH 5. The foaming cleanser may be essentially free or devoid of added PEG-type, PEG/PEO polymeric thickeners.

The present invention provides a new type of alternative natural thickeners to replace PEG based ingredients, particularly but not limited to PEG/PEO thickeners, in cleansing products. During the exploration of PEG alternatives, it was unexpectedly discovered that intermediate to long chain fatty alcohols, which are typically liquid or semi-waxy at room temperature and feature carbon chain lengths from 8 up to 22, can effectively thicken the formulation. These fatty alcohols can be linear or branched, saturated or unsaturated, and may have hydroxyl groups in either terminal or non-terminal positions. Traditionally, fatty alcohols are recognized for their cosmetic properties, including emollient, hydrating, softening, and protective effects. While long chain fatty alcohols (C22 or greater) are often used to impart a waxy texture in hair and skincare products due to their high melting points, it was surprising to find that intermediate chain fatty alcohols (C8 to C22) also serve as effective thickeners in cleansing compositions. These fatty alcohols, although previously known as emollients in facial cleansers, were not recognized for their thickening ability. However, they have been found to significantly enhance the viscosity and sensoriality of the formulation, providing a viable substitute for PEG, while maintaining or even improving foam characteristics.

In some embodiments, the foaming cleanser excludes PEG/PEO ingredients and polymeric thickeners, parabens, and combinations thereof. This exclusion further enhances the safety profile of the cleanser, reducing the risk of adverse skin reactions. The inventive composition demonstrates performance that is at least as effective as, if not improved over, commercial benchmarks and comparatives containing PEG ingredients, providing moisturizing properties without excessive greasiness, demonstrating foaming that is not compromised even in the absence of PEG/PEO, balancing cleansing efficacy with skin hydration, providing versatility in formulation to achieve desired sensory and functional attributes, and enhancing the formulation's safety and appeal for consumers seeking cleaner beauty products. optimizing foaming and cleansing performance.

### Exclusion of PEG-Based Ingredients

A notable feature of the foaming cleanser formulation is the exclusion of PEG-based ingredients, including PEG/PEO polymeric thickeners and parabens. This exclusion addresses concerns related to potential skin irritation and environmental safety, aligning the product with current consumer preferences for cleaner and more natural formulations.

The described foaming cleanser is designed to deliver effective cleansing while maintaining the skin's natural moisture balance and providing a pleasing sensory experience. By selecting ingredients that are both effective and gentle, the formulation caters to a wide range of skin types and consumer needs.

“Cleanser” or “foaming cleanser” as used herein means and refers to any foaming cleanser utilized for application to a keratinous tissue for one or more of cleansing the skin, removal of make-up and the like.

“Cosmetically acceptable” as used herein means and refers to a carrier that is compatible with any keratinous substrate.

As used herein with respect to the foaming property of cleansing formulations, including the foaming cleanser, the term “foaming” means and refers to the capacity of a composition (inventive or comparative) to develop a foam upon rubbing that would be associated with applying and gently scrubbing skin or other keratinous substrates with the formulation. Reference is made to a known testing method for establishing foam formation in vitro employing equipment that is well known in the art, namely analysis using a Krauss DFA100 ™ Dynamic Foam Analyzer, which provides a relative measure of achieved foam formation.

“Keratinous substrate” and “keratinous tissue” each includes but is not limited to skin, hair, and nails.

“Skin” as used herein means and refers to skin materials containing keratin such as facial and body skin, scalp, eyebrows, and lips.

“Stable” and “Stability” as used herein means that the foaming cleanser does not show any signs of significant changes in one or more of color, odor, pH, visible clarity (minimal or no cloudiness or sedimentation or precipitation) or viscosity at room temperature and at temperatures in the range from about 25° C. to about 45° C. In various embodiments, an inventive composition remains stable at temperatures in the range from about 25° C. to about 45° C., over a time period of at least one month, or at least two months, or longer, or any value, range, or sub-range therebetween. In some embodiments, a foaming cleanser is stable at 25° C. for at least one month, or at least two months, or longer, or any value, range, or sub-range there between. In some embodiments, a foaming cleanser is stable at 45° C. for at least one month, or at least two months, or longer, or any value, range, or sub-range therebetween. Viscosity was measured in the UD units by viscometer Rheomat R180 using an M3 spindle for ~10 minutes at 25° C. Stable inventive compositions demonstrate viscosity in a range from about 10 UD to about 40 UD (M3) based on use of RM180 Rheomat M3 Spindle analysis. In some particular embodiments, the viscosity is in the range from about 20 UD to about 30 UD. Unsatisfactory compositions are deemed unstable at viscosities that fall below 10 UD.

In various embodiments, the foaming cleanser may be a skin cleanser, shampoo, or body wash and in some particular embodiments, the foaming cleanser is a facial cleanser and/or makeup remover.

The foaming cleanser, can comprise, consist of, or consist essentially of the essential elements and limitations of the invention described herein, as well as any additional or optional ingredients, components, or limitations described herein or otherwise useful.

Unless otherwise stated, such as for ratios between two ingredients, which are typically based on the weights of each ingredient.

### Foaming Cleanser

### Fatty Alcohols

The foaming cleanser includes at least one fatty alcohol, in particular medium chain length fatty alcohol.

Fatty alcohols used in the cleanser can vary widely, providing flexibility in formulation. These alcohols may be selected from those that are liquid or semi-waxy at room temperature, and they typically contain carbon chain lengths ranging from 6 to 22. This category includes both linear and branched structures, saturated or unsaturated forms, and may feature hydroxyl groups in either terminal or non-terminal positions.

Specific examples of fatty alcohols include hexyldecanol, oleoyl alcohol, lauryl alcohol, myristyl alcohol, cetyl alcohol, decanol, butyloctanol, hexydecanol, and palmitoleyl alcohol. Combinations of these alcohols can be used to achieve desired properties in the foaming cleanser.

In particular embodiments, the fatty alcohol is selected from hexyldecanol, oleoyl alcohol, lauryl alcohol, myristyl alcohol, cetyl alcohol, and combinations thereof. Specific blends, such as a combination of lauryl alcohol, myristyl alcohol, and cetyl alcohol may be selected. The formulation may consist of single alcohols like hexyldecanol or oleoyl alcohol, or it may comprise a blend of these with other fatty alcohols.

In various embodiments, the amount of the at least one fatty alcohol is from about 0.05% to about 2%, or from about 0.05% to about 1%, or from about 0.05% to about 0.8%, or from about 0.05% to about 0.6%, or from about 0.05% to about 0.5%, or from about 0.1% to about 1.5%, or from about 0.1% to about 1%, or from about 0.2% to about 0.8%, or any suitable combination, sub-combination, range, or sub-range thereof by weight, based on the weight of the foaming cleanser. One of ordinary skill in the art, however, will appreciate that other ranges are within the scope of the invention. Thus, fatty alcohols may be present by weight, based on the total weight of the foaming cleanser, from about 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9 to about 2 weight percent, including increments and ranges therein and there between.

### Surfactants

In some embodiments, the foaming cleanser may include or excludes any one or more of amphoteric, ionic, nonionic or cationic surfactants, and in particular embodiments, may excluded sulfate, PEG/POE, or a combination of such surfactants.

### Amphoteric Surfactants

In various embodiments, the foaming cleanser comprises at least one amphoteric surfactants (or zwitterionic surfactant).

In some embodiments, the at least one amphoteric surfactant is present from about 1% to about 15% by weight, based on the weight of the composition. In certain formulations, the amphoteric surfactant is present from about 3% to about 10% by weight.

These surfactants are selected from cocoamidopropyl hydroxysultaine, coco betaine, and cocoamidopropyl betaine, and can be used individually or in combinations.

In some embodiments, the at least one amphoteric surfactant comprises cocoamidopropyl hydroxysultaine.

The amphoteric surfactants possess a carbon chain length distribution from C8-10 up to C16-18, which can overlap with the carbon chain lengths of the fatty alcohols. In some embodiments, the amphoteric surfactants are selected based on overlap of the carbon chain length with that of the fatty alcohol chain length. For example, when each fatty alcohol is liquid at room temperature and contains a carbon chain length from 8 up to 10, the surfactant's chain length distribution from C8-10 complements this. Similarly, when using fatty alcohols with carbon chain lengths from 16 up to 18, amphoteric surfactants with a C16-18 distribution align with the composition.

In various embodiments, the amount of cocoamidopropyl hydroxysultaine may be from about 1% to about 20%, or from about 3% to about 7%, or from about 1% to about 5%, or any suitable combination, sub-combination, range, or sub-range thereof by weight, based on the weight of the foaming cleanser.

In various embodiments, the amount of the at least one amphoteric surfactant is from about 1% to about 15%, or from about 3% to about 10%, or from about 3% to about 8%, or from about 3% to about 6%, or from about 3% to about 5%, or from about 5% to about 12%, or from about 5% to about 10%, or from about 6% to about 8%, or any suitable combination, sub-combination, range, or sub-range thereof by weight, based on the weight of the foaming cleanser. One of ordinary skill in the art, however, will appreciate that other ranges are within the scope of the invention. Thus, amphoteric surfactants may be present by weight, based on the total weight of the foaming cleanser, from about 1, 2, 3, 4, 5, 6, 7, 8, 9, 10, 11, 12, 13, 14 to about 15 weight percent, including increments and ranges therein and there between.

In some embodiments, the foaming cleanser comprises one or more additional amphoteric surfactants.

In some embodiments, one or more additional amphoteric surfactant, if present, may be selected from, for example, betaines, alkyl sultaines, alkyl amphoacetates and alkyl amphodiacetates, alkyl amphoproprionates, amphocarboxylates, alkyl betaines, amidoalkyl betaines, amphophosphates, phosphobetaines, pyrophosphobetaines, carboxyalkyl polyamines, amidoalkyl sultaines, salts thereof, or mixtures thereof.

Betaines which can be used in the current compositions include those having the formulas below:

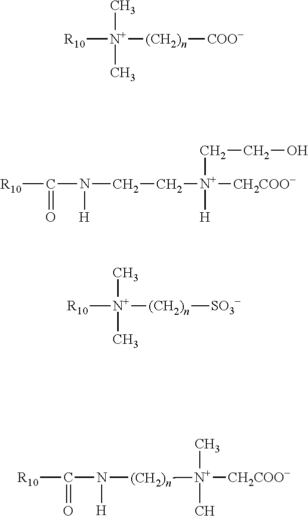

wherein R¹⁰ is an alkyl group having 8-18 carbon atoms; and n is an integer from 1 to 3.

Particularly useful betaines include, for example, coco betaine, cocoamidopropyl betaine, lauryl betaine, laurylhydroxy sulfobetaine, lauryldimethyl betaine, cocoamidopropyl hydroxysultaine, behenyl betaine, capryl/capramidopropyl betaine, lauryl hydroxysultaine, stearyl betaine, and mixtures thereof. Typically, the at least one betaine compound is selected from the group consisting of coco betaine, cocoamidopropyl betaine, behenyl betaine, capryl/capramidopropyl betaine, lauryl betaine, and mixtures thereof, and more typically coco betaine.

Hydroxyl sultaines useful in the compositions of the invention include the following

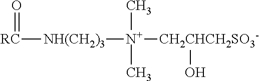

wherein R is an alkyl group having 8-18 carbon atoms.

More specific examples include, but are not limited to cocamidopropyl hydroxysultaine, lauryl hydroxysultaine, or mixtures thereof.

Useful alkylamphoacetates include those having the formula

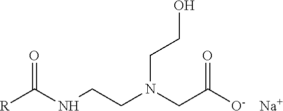

wherein R is an alkyl group having 8-18 carbon atoms.

Useful alkyl amphodiacetates include those having the formula

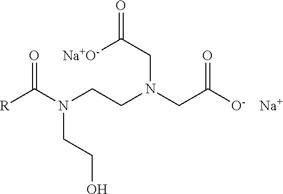

wherein R is an alkyl group having 8-18 carbon atoms.

### Alkyl Amphopropionates

Exemplary and non-limiting examples of useful alkyl amphopropionates include cocoamphopropionate, caprylamphopropionate, cornamphopropionate, caproampho-propionate, oleoamphopropionate, isostearoamphopropionate, stearoamphopropionate, lauroamphopropionate, salts thereof, or mixtures thereof.

The one or more additional amphoteric surfactant of the present disclosure may be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

The one or more additional amphoteric surfactant may be present in the foaming cleanser in a range from about 0.1% to about 20%, based on the weight of the foaming cleanser.

In various embodiments, each of one or more additional amphoteric surfactant may be present in an amount in the range of from about 0.1% to about 20%, and the total amount of surfactant may be present in a range from about 0.1% to about 20%. In some embodiments, each of one or more additional amphoteric surfactant is present in an amount that is from about 1% to about 20%, or from about 1% to about 15%, or from about 3% to about 10%, or from about 5% to about 7%, or any value, range, or sub-range therebetween by weight, based on the weight of the foaming cleanser.

Thus, in various embodiments, each of one or more additional amphoteric surfactant may be present in a composition according to the disclosure from 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 to about 20 percent, including increments and ranges therein and there between.

### Anionic Surfactants

The foaming cleanser includes at least one anionic surfactant. In some embodiments, the at least one anionic surfactant is present from about 1% to about 15% by weight, based on the weight of the composition, and in some embodiments, from about 3% to about 10% by weight.

Anionic surfactants may be selected from sarcosinate, glycinate, glutamate, taurate, isethionate, alpha olefin sulfonate, and combinations thereof.

In some embodiments, the foaming cleanser comprises sodium methyl cocoyl taurate as a preferred anionic surfactant. In various embodiments, the foaming cleanser comprises the anionic surfactant comprising sodium methyl cocoyl taurate.

In some embodiments, the foaming cleanser comprises one or more anionic surfactants in addition to sodium methyl cocoyl taurate, for example, one or more of sodium lauroyl sarcosinate, sodium methyl cocoyl taurate, sodium isethionate and sodium lauroyl lactylate, present from about 0.001% to about 2%, or from about from about 0.005% to about 0.01%, or from about 0.015% to about 0.035%, or from about 0.02% to about 2%.

In various embodiments, the amount of the at least one anionic surfactant is from about 1% to about 15%, or from about 3% to about 10%, or from about 3% to about 8%, or from about 3% to about 6%, or from about 3% to about 5%, or from about 5% to about 12%, or from about 5% to about 10%, or from about 6% to about 8%, or any suitable combination, sub-combination, range, or sub-range thereof by weight, based on the weight of the foaming cleanser. One of ordinary skill in the art, however, will appreciate that other ranges are within the scope of the invention. Thus, anionic surfactants may be present by weight, based on the total weight of the foaming cleanser, from about 1, 2, 3, 4, 5, 6, 7, 8, 9, 10, 11, 12, 13, 14 to about 15 weight percent, including increments and ranges therein and there between.

In some embodiments, the foaming cleanser comprises one or more additional anionic surfactants. More generally, at least one additional anionic surfactant, if present, may be selected from non-sulfate anionic surfactants, sulfate anionic surfactants, their salts thereof, or mixtures thereof.

In some embodiments, the foaming cleanser may comprise among optional additional anionic surfactants or one or more of rhamnolipids; these may be present from about 0% to about 1%, or from about 0.5% to about 0.5%, or polylysine present from about 0% to about 1%, or from about 0.005% to about 0.0.1%, or a combination thereof, the amounts representing an exemplary embodiment, by weight, based on the weight of the composition.

### Non-Sulfate Anionic Surfactants

When present, the total amount of non-sulfate anionic surfactants in the foaming cleansers can vary but typically ranges from about 4% to about 30%, based on the total weight of the foaming cleanser. In some cases, the total amount of non-sulfate anionic surfactants in the foaming cleanser may be from about 4% to about 25%, or from about 5% to about 25%, or from about 5% to about 23%, or from about 6% to about 24%, or from about 6% to about 25%, or from about 7% to about 25%, or from about 8% to about 25%, or from about 10% to about 23%, or any suitable combination, sub-combination, range, or sub-range thereof by weight, based on the weight of the foaming cleanser based.

In various embodiments, the total amount of non-sulfate anionic surfactants in the foaming cleansers, if present, is typically at about 6, 6.5, 7, 7.5, 8, 8.5, 8.8, 9, 9.5, 10, 10.5, 11, 11.4, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, to about 25, weight percent based on the total weight of the foaming cleanser, including increments and ranges therein and there between.

Useful non-sulfate anionic surfactants include, but are not limited to, alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, or mixtures thereof. Non-limiting examples of useful non-sulfate anionic surfactants are provided below.

### Acyl Isethionates

Non-limiting examples of useful acyl isethionates and their salts include those of formula (III) and (IV):

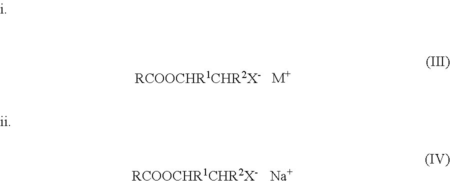

wherein R, R¹, and R² are each independently chosen from H or an alkyl chain having 1-24 carbon atoms, said chain being saturated or unsaturated, linear or branched; X is COO⁻ or SO₃⁻; and M is any suitable cation.

Although the cation may be chosen from any suitable cation as in formula (III), including, for example, alkali metal ion such as sodium or potassium, ammonium ions, or alkanolammonium ions such as monoethanolammonium or triethanolammonium ions, sodium is an cation, as in formula (IV). In various embodiments, RCO— represents the coconut acid moiety. Non-limiting examples of acyl isethionates include sodium cocoyl isethionate, sodium lauroyl isethionate, sodium lauroyl methyl isethionate, and sodium cocoyl methyl isethionate.

### Acyl Sarcosinates

Non-limiting examples of acyl sarcosinates and their salts include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

In some embodiments, the total amount of acyl sarcosinates in the foaming cleanser, if present, is from about 0.5 to about 10%, all amounts by weight based on the weight of the foaming cleanser. In some instance, the total amount of acyl sarcosinates in the foaming cleanser is from about 0.5 to about 10%, about 1 to about 8%, about 1.5 to about 7%, about 1.75 to about 6%, 2 to about 5%, about 2 to about 4%, about 2.5 to about 4%, about 2.5 to about 3.5%, or about 2.7 to about 3%, all amounts by weight based on the weight of the foaming cleanser, including all ranges and subranges therebetween.

### Alkyl Sulfonates

Useful alkyl sulfonates and their salts include alkyl aryl sulfonates, primary alkane disulfonates, alkene sulfonates, hydroxyalkane sulfonates, alkyl glyceryl ether sulfonates, alpha-olefinsulfonates, sulfonates of alkylphenolpolyglycol ethers, alkylbenzenesulfonates, phenylalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

In some instances, an alkyl sulfonate of formula (V) is particularly useful:

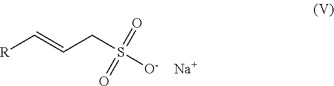

wherein R is selected from H or alkyl chain that has 1-24 carbon atoms, 6-24 carbon atoms, more, 8 to 20 carbon atoms, said chain being saturated or unsaturated, linear or branched, substituted or unsubstituted. Sodium is shown as the cation in the above formula (V) but the cation may be an alkali metal ion such as sodium or potassium, ammonium ions, or alkanolammonium ions such as monoethanolammonium or triethanolammonium ions.

In some instances, the alkyl sulfonate(s) are selected from C₈-C₁₆ alkyl benzene sulfonates, C₁₀-C₂₀ paraffin sulfonates, C₁₀-C₂₄ olefin sulfonates, salts thereof, or mixtures thereof. A non-limiting but particularly useful example of a C₁₀-C₂₄ olefin sulfonate that can be used in the instant compositions is sodium C₁₄₋₁₆ olefin sulfonate.

### Alkyl Sulfosuccinates

Non-limiting examples of useful alkyl sulfosuccinates and their salts include those of formula (VI):

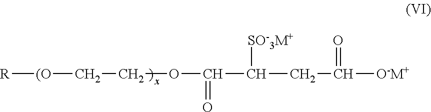

wherein:
1. R is a straight or branched chain alkyl or alkenyl group having 10 to 22 carbon atoms, 10 to 20 carbon atoms;
2. x is a number that represents the average degree of ethoxylation, and can range from 0 to about 5, from 0 to about 4, and most from about 2 to about 3.5; and
3. M, which can be the same or different, is chosen from any suitable monovalent cation.

Preferred cations are alkali metal ions such as sodium or potassium, ammonium ions, or alkanolammonium ions such as monoethanolammonium or triethanol-ammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or mixtures thereof.

### Alkyl Sulfoacetates

Non-limiting examples of alkyl sulfoacetates and their salts include, for example, alkyl sulfoacetates such as C4-C18 fatty alcohol sulfoacetates and/or salts thereof, for example, sodium lauryl sulfoacetate. Useful cations for the salts include alkali metal ions such as sodium or potassium, ammonium ions, or alkanolammonium ions such as monoethanolammonium or triethanolammonium ions.

### Alkoxylated Monoacids

Non-limiting examples of alkoxylated monoacids include compounds corresponding to formula (VII):

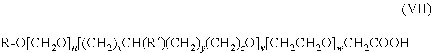

wherein:
1. R is a hydrocarbon radical containing from about 6 to about 40 carbon atoms;
2. R′ represents hydrogen or alkyl;
3. u, v, and w, which may be identical or different, independently represent numbers from 0 to 60;
4. x, y, and z, which may be identical or different, independently represent numbers from 0 to 13; and
5. the sum of x+y+z>0.

Compounds corresponding to formula (VII) can be obtained by alkoxylation of alcohols R—OH with ethylene oxide as the sole alkoxide or with several alkoxides and subsequent oxidation. The numbers u, v, and w each represent the degree of alkoxylation. Whereas, on a molecular level, the numbers u, v, and w and the total degree of alkoxylation can only be integers, including zero, on a macroscopic level they are mean values in the form of broken numbers.

In formula (VII), R is linear or branched, acyclic or cyclic, saturated or unsaturated, aliphatic or aromatic, substituted or unsubstituted. Typically, R is a linear or branched, acyclic C6-C40 alkyl or alkenyl group or a C1-C40 alkyl phenyl group, more typically a C8-C22 alkyl or alkenyl group, or a C4-C18 alkyl phenyl group, and even more typically a C12-C18 alkyl group or alkenyl group or a C6-C16 alkyl phenyl group. Further, u, v, w, independently of one another, are typically chosen from a number ranging from 2 to 20, more typically a number ranging from 3 to 17, and most typically a number ranging from 5 to 15. Further still, x, y, z, independently of one another, are typically chosen from a number ranging from 0 to 13, more typically a number ranging from 1 to 10, and most typically a number ranging from 2 to 8.

Suitable alkoxylated monoacids include, but are not limited to: Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C11-15 Pareth-7 Carboxylic Acid, C12-13 Pareth-5 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C14-15 Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid, or mixtures thereof.

### Acyl Amino Acids

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt.

Non-limiting examples of useful acyl amino acids include those of formula (VIII):

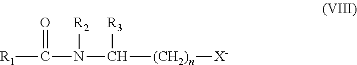

wherein: R¹, R², and R³ are each independently selected from H or an alkyl chain having 1-24 carbon atoms, said chain being saturated or unsaturated, linear or branched, substituted or unsubstituted; n ranges from 0 to 30; and X is COO⁻ or SO₃⁻.

In various embodiments, RCO— represents the coconut acid moiety. A non-limiting example is sodium cocoyl glycinate.

### Acyl Taurates

Non-limiting examples of acyl taurates include those of formula (IX):

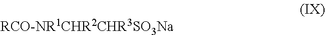

wherein R, R¹, R², and R³ are each independently selected from H or an alkyl chain having from 1-24 carbon atoms, such as from 6-20 carbon atoms, or from 8-16 carbon atoms, said chain being saturated or unsaturated, linear or branched, substituted or unsubstituted.

In various embodiments, RCO— represents the coconut acid moiety. Non-limiting examples of acyl taurate salts include sodium cocoyl taurate and sodium methyl cocoyl taurate.

### Acyl Glycinates

Non-limiting examples of useful acyl glycinates include those of formula (X):

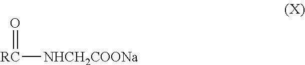

wherein R is an alkyl chain of 8 to 16 carbon atoms. Sodium is shown as the cation in the above formula (X), but the cation may be any alkali metal ion such as sodium or potassium, ammonium ions, or alkanolammonium ions such as monoethanolammonium or triethanolammonium ions.

Non-limiting examples of acyl glycinates include sodium cocoyl glycinate, sodium lauroyl glycinate, sodium myristoyl glycinate, potassium lauroyl glycinate, and potassium cocoyl glycinate, and in particular sodium cocoyl glycinate.

### Acyl Glutamates

Non-limiting examples of useful acyl glutamates include those of formula (XI):

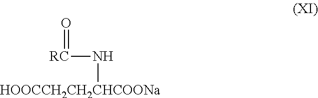

wherein R is an alkyl chain of 8 to 16 carbon atoms. Sodium is shown as the cation in the above formula (XI) but the cation may be any alkali metal ion such as sodium or potassium, ammonium ions, or alkanolammonium ions such as monoethanolammonium or triethanolammonium ions.

Non-limiting examples of acyl glutamates include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate.

In an embodiment, the non-sulfate anionic surfactants of the foaming cleansers of the present disclosure may be selected from one or more acyl isethionates, their salts, or mixtures thereof.

In an embodiment, the one or more acyl isethionates, their salts, or mixtures thereof are selected from sodium cocoyl isethionate, sodium lauroyl methyl isethionate, sodium cocoyl methyl isethionate, sodium lauroyl isethionate, or mixtures thereof.

In an embodiment, the non-sulfate anionic surfactants of the foaming cleansers of the present disclosure are selected from one or more acyl sarcosinates, their salts, or mixtures thereof.

In an embodiment, the one or more acyl sarcosinates, their salts, or mixtures thereof are selected from sodium cocoyl sarcosinate, sodium lauroyl sarcosinate, potassium lauroyl sarcosinate, potassium cocoyl sarcosinate, sodium myristoyl sarcosinate, sodium oleoyl sarcosinate, sodium palmitoyl sarcosinate, ammonium lauroyl sarcosinate, or mixtures thereof.

For example, two or more, such as three or more non-sulfate anionic surfactants may be selected from acyl isethionates, acyl sarcosinates, alkyl sulfonates, alkyl sulfosuccinates, alkyl sulfoacetates, alkoxylated monoacids, and acyl amino acids such as acyl taurates, acyl glycinates, acyl glutamates, salts thereof, or mixtures thereof.

### Sulfate Anionic Surfactants

Optionally, anionic surfactants may be chosen from sulfate anionic surfactants. Such sulfate anionic surfactants may be chosen from, for example, alkyl sulfates, alkyl ether sulfates, and/or salts thereof.

By way of example, alkyl sulfates may include C₈₋₁₈ alkyl sulfates, more C₁₂₋₁₈ alkyl sulfates, such as in the form of a salt with a solubilizing cation such as sodium, potassium, ammonium, or substituted ammonium. Examples include but are not limited to sodium lauryl sulfate (SLS) and sodium dodecyl sulfate (SDS).

As a further example, alkyl ether sulfates may include those having the formula:

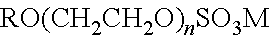

wherein:
R is an alkyl or alkenyl having from 8 to 18 (12 to 18) carbon atoms;
n is a number having an average value of greater than at least 0.5, between 1 and 3, more between 2 and 3; and
M is a solubilizing cation such as sodium, potassium, ammonium or substituted ammonium.

An example of a useful alkyl ether sulfate is sodium lauryl ether sulfate (SLES).

In some instances, useful alkyl sulfate salts and alkyl ether sulfate salts include those having the formulas (XII and XIII):

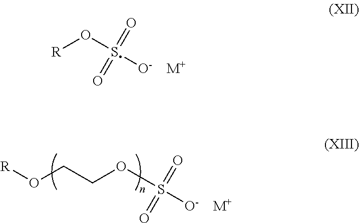

Wherein R is an alkyl chain having from 6 to 24 carbon atoms, 8 to 18 carbon atoms, and more 12 to 18 carbon atoms; M is a solubilizing cation such as alkali metal ions such as sodium or potassium, ammonium ions, or alkanolammonium ions such as monoethanolammonium or triethanolammonium ions; and n is an integer from 0 to 3.

In some embodiments, foaming cleanser according to the disclosure does not require silicones and/or sulfate-based anionic surfactants. Thus, any one or more (or all) of these may optionally be excluded from the foaming cleansers. In other words, the foaming cleansers may be free or essentially free of silicones and/or sulfate-based anionic surfactants. Nonetheless, in some instances, one or more silicones and/or one or more sulfate-based surfactants may optionally be included in at least certain embodiments of the foaming cleansers.

Thus, in various embodiments, one or more additional anionic surfactant may be present in a composition according to the disclosure from 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 120, 21, 22, 23, 24, 25, 26, 27, 28, 29, 30, 3, 32, 33, 34, 35, 36, 37, 38, 39, 40, 41, 42, 43, 44, 45, 46, 47, 48, 49 to about 50 percent, including increments and ranges therein and there between.

### Water

In the various embodiments, the foaming cleanser includes water.

In various embodiments, the foaming cleanser comprises from about 40% to about 90% water, and In some embodiments, from about 50% to about 85% water, and In some embodiments, from about 70% to about 80% water, including increments and all ranges and subranges therein and there between, by weight, based on the total weight of the foaming cleanser. One of ordinary skill in the art, however, will appreciate that other ranges are within the scope of the invention. Thus, water is present, by weight, based on the total weight of the foaming cleanser, from about 40, 45, 50, 55, 60, 65, 66, 67, 68, 69, 70, 71, 72, 73, 74, 75, 80, 85, to about 90 weight percent, including increments and all ranges and subranges therein and there between. In some embodiments, water is present at about 74%.

The water used in the foaming cleanser may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

### Additional Ingredients

In addition to the foregoing, the composition may also incorporate various additional ingredients that enhance its properties and performance.

In some embodiments, the composition may include one or more of other additional ingredients (also referred to as an additive or active) selected from the group consisting of skin care actives, humectants, conditioning agents, thickeners, viscosity adjusters, cooling agents, fillers, antimicrobials, preservatives, pH adjusters, chelating agents, surfactants (anionic, amphoteric, cationic, nonionic), emulsifiers, oils, and combinations thereof, wherein the one or more of other optional ingredients does not adversely affect the properties of the foaming cleanser according to the invention.

The foaming cleanser may further include additional ingredients. These additional ingredients can include niacinamide, phenoxyethanol, sodium hyaluronate, glycerin, sodium chloride, sodium benzoate, sodium lauroyl lactylate, ceramide NP, ceramide AP, phytosphingosine, cholesterol, xanthan gum, carbomer, ceramide EOP, propanediol, tetrasodium glutamate diacetate, and citric acid. Each of these components serves a specific function, such as moisturizing, preserving, or stabilizing the product. They can be selected and combined based on the desired characteristics of the final product.

In some embodiments, the foaming cleanser may include an additional ingredient selected from the group consisting of niacinamide, phenoxyethanol, water/aqua, sodium hyaluronate, glycerin, sodium chloride, sodium benzoate, sodium lauroyl lactylate (and) ceramide NP (and) ceramide AP (and) phytosphingosine (and) cholesterol (and) xanthan gum (and) carbomer (and) ceramide EOP, propanediol, tetrasodium glutamate diacetate, citric acid, and combinations thereof.

In some embodiments, the foaming cleanser may include an additional ingredient selected from the group consisting of niacinamide (0%-5.00%), phenoxyethanol (0%-2%), sodium hyaluronate (0%-1%), glycerin (0%-20%), sodium chloride (0%-5%), sodium benzoate (0%-1%), sodium lauroyl lactylate (and) ceramide NP (and) ceramide AP (and) phytosphingosine (and) cholesterol (and) xanthan gum (and) carbomer (and) ceramide EOP (0%-1%), propanediol (0%-20%), tetrasodium glutamate diacetate (0%-(0.5%), citric acid (0%-1%), PEG-150 pentaerythrityl tetrastearate (0%), and combinations thereof, wherein the additional ingredient may be present in the range stated herein, all amounts by weight, based on the weight of the composition.

In some embodiments, the foaming cleanser includes additional ingredient comprising niacinamide (~2%), phenoxyethanol (~0.5%), sodium hyaluronate (~0.001%), glycerin (~5%), sodium chloride (~1.5%), sodium benzoate (~0.3%), sodium lauroyl lactylate (and) ceramide NP (and) ceramide AP (and) phytosphingosine (and) cholesterol (and) xanthan gum (and) carbomer (and) ceramide EOP (~0.25%), propanediol (~3%), tetrasodium glutamate diacetate (~0.1%), citric acid (~0.4%), wherein the additional ingredient may be present in the amount stated herein, all amounts by weight, based on the weight of the composition.

In various embodiments, additional ingredients may be present in amounts ranging from about 0.0001% to about 20%, or from about 0.01% to about 10%, or from about 0.1% to about 5%, or from about 0.5% to about 2%, or from about 1% to about 3%, or any suitable combination, sub-combination, range, or sub-range thereof by weight, based on the weight of the composition. One of ordinary skill in the art, however, will appreciate that other ranges are within the scope of the invention. Thus, additional ingredients may be present by weight, based on the total weight of the composition, from about 0.0001, 0.001, 0.01, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 to about 20 weight percent, including increments and ranges therein and there between.

Although the aforementioned optional components are given as an example, it will be appreciated that other optional components compatible with cosmetic applications known in the art may be used.

In some embodiments, the composition includes at least one additive used in the cosmetics field which does not adversely affect the properties of the composition according to the invention, such as, water soluble solvents, oils, preservatives, as well as fragrances, pH adjusters (citric acid, sodium chloride, lactic acid); neutralizing or pH-adjusting agents (e.g., triethylamine (TEA) and sodium hydroxide, Trisodium ethylenediamine disuccinate, and combinations thereof), other cosmetically acceptable additives, such as but not limited to, pearlescent agents, silica, and coloring materials; essential oils; fruit extracts, for example, Pyrus Malus (Apple) Fruit Extract, and Aloe Barbadensis Leaf Juice Powder; actives (for example, hydroxyacetophenone); vitamins (for example, vitamin A, beta carotene, tocopherol/vitamin E, panthenol, retinol, resveratrol, vitamin C, niacinamide, derivatives thereof, and combinations thereof); hyaluronic acid in the form of hydrolyzed hyaluronic acid or sodium hyaluronate; coloring materials/pigments; essential oils; antioxidants (phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria baicalensis root extract), pine bark extract (Pinus pinaster bark/bud extract), ellagic acid); vitamins and vitamin derivatives, such as calcium pantothenate, tocopherol and ascorbic acid; hydroxy acids; citric acid, sodium citrate, sodium chloride; neutralizing, chelating or pH-adjusting agents (for example, triethylamine (TEA), trisodium ethylenediamine disuccinate, EDTA, and sodium hydroxide); powders, fragrances, dyes, pigments; organic or mineral UV filters; conditioning agents such as C12-15 Alkyl lactate and C12-15 Alcohols; lauryl lactate, Thickeners, for example Ammonium polyacryloyldimethyl taurate, Propylene glycol and PEG-55 propylene glycol oleate; viscosity adjusters, for example, Sodium chloride and Hexylene Glycol; cooling agents such as menthol; fillers, for example Kaolin; or any combination thereof.

In some embodiments, there may be one or more actives selected from, for example, preservatives/anti-microbials (for example, chlorphenesin, salicylic acid, phenoxyethanol, potassium sorbate, and caprylyl glycol); actives (for example, hydroxyacetophenone); vitamins (for example, vitamin A, beta carotene, tocopherol/vitamin E, panthenol, retinol, resveratrol, vitamin C, niacinamide, derivatives thereof, and combinations thereof); hyaluronic acid in the form of hydrolyzed hyaluronic acid or sodium hyaluronate; coloring materials/pigments; essential oils; antioxidants (phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria baicalensis root extract), pine bark extract (Pinus pinaster bark/bud extract), ellagic acid); vitamins and vitamin derivatives, such as calcium pantothenate, tocopherol and ascorbic acid; hydroxy acids; citric acid, sodium citrate, sodium chloride; neutralizing, chelating or pH-adjusting agents (for example, triethylamine (TEA), trisodium ethylenediamine disuccinate, EDTA, and sodium hydroxide); powders, fragrances, dyes, pigments; organic or mineral UV filters; and combinations thereof.

In the various embodiments, the amount of one or more additives, alone or in combination, present in the composition can be present in a range from about 0.0001% to about 20%, and in some embodiments, from about 0.005% to about 0.01%, and in some embodiments, from about 0.01% to about 0.1%, and in some embodiments, from about 0.15% to about 5%, and in some embodiments, from about 0.40% to about 4%, and in some embodiments, from about 0.5% to about 2.5%, and in some embodiments, from about 0.1% to about 0.5% and in some embodiments, from about 1% to about 2%, or any suitable combination, sub-combination, range, or sub-range thereof by weight, based on the total weight of the composition. One of ordinary skill in the art, however, will appreciate that other ranges are within the scope of the invention.

Thus, any one or a combination of additional ingredients may be present, by weight, based on the total weight of the composition, each one or the combination present from about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 weight percent, including increments and ranges therein and there between.

### Water-Soluble Solvents

In some embodiments, the composition may include as additional ingredients at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions. Non-limiting examples of water-soluble solvents include, for example, glycerin, alcohols (for example, C1-C30, C1-C15, C1-C10, or C₁-C₄ alcohols), polyols, glycols, and combinations thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and combinations thereof.

In the various embodiments total amount of the at least one water-soluble solvent, when present, may vary, is from about 0.5% to about 25%, or from about 0.5% to about 20%, or from about 1% to about 20%, or from about 1% to about 10%, or from about 2% to about 5% or any suitable combination, sub-combination, range, or sub-range thereof by weight, based on the weight of the composition. One of ordinary skill in the art, however, will appreciate that other ranges are within the scope of the invention.

Thus, any one water-soluble solvent, when present, is present, by weight, based on the total weight of the composition, from about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

The pH of the composition is not limited but is generally from 2 and 12, and in some embodiments, is one of from 3 and 11, and from 4 and 9, and from 4.5 and 6, and in some embodiments, is about pH 5.

The pH can be adjusted to the desired value by addition of a base (organic or inorganic) to the composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid.

In some embodiments, the composition excludes alcohols, for example, monoalcohols such as monohydric C₁-C₈ alcohols such as ethanol, propanol, butanol, isopropanol, isobutanol, and benzyl alcohol, and phenylethyl alcohol.

### Oils

In some embodiments, the composition may include as additional ingredients one or more oils. In some embodiments, the composition is essentially free, free of or devoid of oils.

In some examples, the oils may be chosen from hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the composition may include a synthetic oil selected from the group consisting of hydrogenated polyisobutene and hydrogenated polydecene. A hydrocarbon-based oil may be a non-volatile hydrocarbon-based, such as:
(i) hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, musk rose oil, and essential oils, such as Helianthus annuus Seed Oil, Lavandula angustifolia (lavender) Oil, Mentha piperita Oil, Rosmarinus officinalis (rosemary) Leaf Oil Pelargonium graveolens flower oil, Citrus aurantium dulcis (orange) peel oil, Menthe Viridis (spearmint) leaf oil, Citrus aurantifolia (lime) oil, Melaleuca alternifolia (tea tree) leaf oil, Citrus grandis (grapefruit) peel oil, Citrus medica Limonum (lemon) peel oil, rose flower oil, Eucalyptus globulus leaf oil, and combinations thereof.

(ii) synthetic ethers containing from 10 to 40 carbon atoms;

(iii) linear or branched hydrocarbons of mineral or synthetic origin, such as petroleum jelly, polydecenes, hydrogenated polyisobutene such as Parleam, and 40 squalane;

(iv) synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms on condition that R+R′ is ÿ 10, for instance Purcellin oil (cetearyl octanoate), isopropyl myristate, isopropyl palmitate, C12-C15 alkyl benzoate, such as the product sold under the trade name Finsolv TN™ or Witconol TN™ by Witco or Tegosoft TN™ by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name of “Dub Dis” by Stearinerie Dubois, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C12-C13 alkyl) tartrates, such as those sold under the name Cosmacol ETI™ by Enichem Augusta Industriale, and also di(linear C14-C15 alkyl) tartrates, such as those sold under the name Cosmacol ETL™ by the same company; or acetates;
(v) fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
(vi) higher fatty acids, such as oleic acid, linoleic acid or linolenic acid;
(vii) carbonates, such as dicaprylyl carbonate, such as the product sold under the name Cetiol CC™ by Cognis;
(viii) fatty amides, such as isopropyl N-lauroyl sarcosinate, such as the product sold under the trade name Eldew SL 205™ from Ajinomoto; and
(ix) essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

In some embodiments, the composition may include at least one branched or linear liquid alkane with carbon chain length of C11 to C20. In various embodiments, liquid alkanes may be selected from those with a carbon chain length of from C11 to C20. The liquid alkanes may be selected from those with a carbon chain length of from C11 to C20, or from C15 to C19, or one of C11, C12, C13, C14, C15, C16, C17, C18 to C19. In some particular embodiments, suitable liquid alkanes that may be used according to the disclosure include hydrocarbon-based oils containing from 8 to 16 carbon atoms, and especially branched C8-C16 alkanes such as C8-C16 isoalkanes. In some exemplary embodiments, such liquid alkanes may be chosen from isoparaffins, for instance isododecane (also known as 2,2,4,4,6-pentamethylheptane), isodecane, and isohexadecane.

The amount of each of the at least one branched or linear liquid alkane may be present in the composition in a range of from about 1% to about 35% by weight, or from about 15% to about 30% by weight, or from about 10% to about 20% or any suitable combination, sub-combination, range, or sub-range thereof by weight, based on the weight of the composition. One of ordinary skill in the art, however, will appreciate that other ranges are within the scope of the invention.

Thus, each of the at least one branched or linear liquid alkane in the composition may be present by weight, based on the total weight of the composition, from about 1, 2, 3, 4, 5, 6, 7, 8, 9, 10, 11, 12, 13, 14, 15, 16, 17, 128, 19, 20, 21, 22, 23, 24, 25, 26, 27, 28, 29, 30, 31, 32, 33, 34, to about 35 percent, including increments and ranges therein and there between.

### Preservatives

In some embodiments, the composition may include as additional ingredients one or more preservatives and/or antimicrobials.

Any preservative commonly used in cleansing compositions is an acceptable preservative for the composition s herein, such as phenoxyethanol, members from the paraben family such as the methyl, ethyl, propyl, butyl or isobutyl parabens, 4-hydroxy benzoic acid, benzoic acid, sorbic acid, dehydroacetic acid, triclosan, benzyl alcohol, chlorophenesin, or salicylic acid, for example. At more concentrated amounts of suitable solvents for optional additives, in particular, suitable solvents for antimicrobials and preservatives, members from the paraben family may be used as a preservative.

In some embodiments, the one or more preservatives, when present, may be selected from phenoxyethanol, caprylyl glycol, Myrtrimonium bromide, hydroxyacetophenone, ethylhexyl glycerin, chlorphenesin, cetrimonium chloride, hexyl glycerin, octylglycerin, benzylglycerin, 3-heptoyl-2,2-propandiol, and 1,2-hexandiol; polyaminopropyl biguanide, also known as polyhexamethylene biguanide, or PHMB, and combinations thereof. In some embodiments, the composition is free or essentially free of one or more of caprylyl glycol, phenoxyethanol, hexyl glycerin, ethylhexylglycerin, octylglycerin, benzylglycerin, 3-heptoyl-2,2-propandiol, and 1,2-hexandiol; polyaminopropyl biguanide, also known as polyhexamethylene biguanide, or PHMB.

In some embodiments, the preservative may comprise one or more of preservatives selected from the group consisting of organic acids, parabens, formaldehyde donors, phenol derivatives, quaternary ammoniums, alcohols, isothiazolinones, and combinations thereof. Preservatives having antibacterial activity are optionally present in the composition s of the present invention. Examples of organic acid preservatives include, but are not limited to, sodium benzoate, potassium sorbate, benzoic acid and dehydroacetic acid, sorbic acid, and combinations thereof. A preferred organic acid preservative system includes a mixture of sodium benzoate and potassium sorbate. Examples of paraben preservatives include, but are not limited to, alkyl para-hydroxybenzoates, wherein the alkyl radical has from 1, 2, 3, 4, 5 or 6 carbon atoms and for example, from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben). Examples of formaldehyde donor preservatives include, but are not limited to, 1,3-Dimethylol-5,5-dimethylhydantoin (DMDM hydantoin), imidazolidinyl urea, gluteraldehyde, and combinations thereof. Examples of quaternary ammonium preservatives include, but are not limited to, benzalkonium chloride, methene ammonium chloride, benzethonium chloride, and combinations thereof. Examples of alcohol preservatives include, but are not limited to, ethanol, benzyl alcohol, dichlorobenzyl alcohol, phenoxyethanol, and combinations thereof. Examples of isothiazolone preservatives include, but are not limited to, methylchloroisothiazolinone, methylisothiazolinone, and combinations thereof.

In some embodiments, the preservative includes one or more preservatives, the one or combination present at a concentration, by weight of about 0.001% to about 5%, or alternatively about 0.05% to about 2.5% or alternatively about 0.1% to about 2.0%, or from about 0.02% to about 1% by weight, or from about 0.03% to about 0.5%, or any suitable combination, sub-combination, range, or sub-range thereof by weight, based upon weight of the composition. One of ordinary skill in the art, however, will appreciate that other ranges are within the scope of the invention.

Thus, any one of or a combination of preservatives, when present, may be present, by weight, based on the total weight of the composition, is from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2, 3, 4, to about 5 weight percent, including increments and ranges therein and there between.

### Cationic Polymers/Polyquaterniums

In some embodiments, the composition may include as additional ingredients one or more polyquaternium cationic polymers.

Generally, the composition may include one or more polyquaternium cationic polymers, also referred to as “polyquaterniums.” For example, the hair treatment composition may include polyquaternium-1 (ethanol, 2,2′,2″-nitrilotris-, polymer with 1,4-dichloro-2-butene and N,N,N′,N′-tetramethyl-2-butene-1,4-diamine), polyquaternium-2, (poly[bis(2-chloroethyl) ether-alt-1,3-bis[3-(dimethylamino)propyl]urea]), polyquaternium-4, (hydroxyethyl cellulose dimethyl diallylammonium chloride copolymer; Diallyldimethylammonium chloride-hydroxyethyl cellulose copolymer), polyquaternium-5 (copolymer of acrylamide and quaternized dimethylammoniumethyl methacrylate), polyquaternium-6 (poly(diallyldimethylammonium chloride)), polyquaternium-7 (copolymer of acrylamide and diallyldimethylammonium chloride), polyquaternium-8 (copolymer of methyl and stearyl dimethylaminoethyl ester of methacrylic acid, quaternized with dimethylsulphate), polyquaternium-9 (homopolymer of N,N-(dimethylamino)ethyl ester of methacrylic acid, quaternized with bromomethane), polyquaternium-10 (quaternized hydroxyethyl cellulose), polyquaternium-11 (copolymer of vinylpyrrolidone and quaternized dimethylaminoethyl methacrylate), polyquaternium-12 (ethyl methacrylate/abietyl methacrylate/diethylaminoethyl methacrylate copolymer quaternized with dimethyl sulfate), polyquaternium-13 (ethyl methacrylate/oleyl methacrylate/diethylaminoethyl methacrylate copolymer quaternized with dimethyl sulfate), polyquaternium-14 (trimethylaminoethylmethacrylate homopolymer), polyquaternium-15 (acrylamide-dimethylaminoethyl methacrylate methyl chloride copolymer), polyquaternium-16 (copolymer of vinylpyrrolidone and quaternized vinylimidazole), polyquaternium-17 (adipic acid, dimethylaminopropylamine and dichloroethylether copolymer), polyquaternium-18 (azelanic acid, dimethylaminopropylamine and dichloroethylether copolymer), polyquaternium-19 (copolymer of polyvinyl alcohol and 2,3-epoxypropylamine), polyquaternium-20 (copolymer of polyvinyl octadecyl ether and 2,3-epoxypropylamine), polyquaternium-22 (copolymer of acrylic acid and diallyldimethylammonium chloride), polyquaternium-24 (auaternary ammonium salt of hydroxyethyl cellulose reacted with a lauryl dimethyl ammonium substituted epoxide), polyquaternium-27 (block copolymer of polyquaternium-2 and polyquaternium-17), polyquaternium-28 (copolymer of vinylpyrrolidone and methacrylamidopropyl trimethylammonium), polyquaternium-29 (chitosan modified with propylen oxide and quaternized with epichlorhydrin), polyquaternium-30 (ethanaminium, N-(carboxymethyl)-N,N-dimethyl-2-[(2-methyl-1-oxo-2-propen-1-yl)oxy]-, inner salt, polymer with methyl 2-methyl-2-propenoate), polyquaternium-31 (N,N-dimethylaminopropyl-N-acrylamidine quaternized with diethylsulfate bound to a block of polyacrylonitrile), polyquaternium-32 (poly(acrylamide 2-methacryloxyethyltrimethyl ammonium chloride)), polyquaternium-33 (copolymer of trimethylaminoethylacrylate salt and acrylamide), polyquaternium-34 (copolymer of 1,3-dibromopropane and N,N-diethyl-N′,N′-dimethyl-1,3-propanediamine), polyquaternium-35 (methosulphate of the copolymer of methacryloyloxyethyltrimethylammonium and of methacryloyloxyethyldimethylacetylammonium), polyquaternium-36 (copolymer of N,N-dimethylaminoethylmethacrylate and buthylmethacrylate, quaternized with dimethylsulphate), polyquaternium-37 (poly(2-methacryloxyethyltrimethylammonium chloride)), polyquaternium-39 (terpolymer of acrylic acid, acrylamide and diallyldimethylammonium Chloride), polyquaternium-42 (poly[oxyethylene(dimethylimino)ethylene (dimethylimino)ethylene dichloride]), polyquaternium-43 (copolymer of acrylamide, acrylamidopropyltrimonium chloride, 2-amidopropylacrylamide sulfonate and dimethylaminopropylamine), polyquaternium-44 (3-Methyl-1-vinylimidazolium methyl sulfate-N-vinylpyrrolidone copolymer), polyquaternium-45 (copolymer of (N-methyl-N-ethoxyglycine)methacrylate and N,N-dimethylaminoethylmethacrylate, quaternized with dimethyl sulphate), polyquaternium-46 (terpolymer of vinylcaprolactam, vinylpyrrolidone, and quaternized vinylimidazole), polyquaternium-47 (terpolymer of acrylic acid, methacrylamidopropyl trimethylammonium chloride, and methyl acrylate), and/or polyquaternium-67.

In some instances, the composition includes one or more polyquaternium cationic polymers selected from cationic cellulose derivatives, quaternized hydroxyethyl cellulose (e.g., polyquaternium-10), cationic starch derivatives, cationic guar gum derivatives, copolymers of acrylamide and dimethyldiallyammonium chloride (e.g., polyquaternium-7), polyquaterniums, and a mixture thereof. For example, the cationic polymer(s) may be selected from polyquaterniums, for example, polyquaterniums selected from polyquaternium-4, polyquaternium-5, polyquaternium-6, polyquaternium-7, polyquaternium-10, polyquaternium-22, polyquaternium-37, polyquaternium-39, polyquaternium-47, polyquaternium-53, polyquaternium-67 and a mixture thereof. A combination of two or more polyquaterniums can be useful. A particularly preferred and useful cationic polymer is polyquaternium-10.

In certain embodiments, the polyquaterniums may be selected from polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-21, polyquaternium-22, polyquaternium-23, polyquaternium-24, polyquaternium-25, polyquaternium-26, polyquaternium-27, polyquaternium-28, polyquaternium-29, polyquaternium-30, polyquaternium-40, polyquaternium-41, polyquaternium-42, polyquaternium-43, polyquaternium-44, polyquaternium-45, polyquaternium-46, polyquaternium-47, polyquaternium-48, polyquaternium-49, polyquaternium-50, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-61, polyquaternium-62, polyquaternium-63, polyquaternium-64, polyquaternium-65, polyquaternium-66, polyquaternium-67, etc. In some cases, preferred polyquaternium compounds include polyquaternium-10, polyquaternium-11, polyquaternium-67, and a mixture thereof.

The total amount of the any polyquaternium cationic polymers, if present, may vary from about 0.01 to about 3 wt. %, based on the total weight of the composition, or from about 0.01 to about 2 wt. %, about 0.01 to about 1 wt. %, about 0.05 to about 3 wt. %, about 0.05 to about 2 wt. %, about 0.05 to about 1 wt. %, about 0.1 to about 3 wt. %, about 0.1 to about 2 wt. %, about 0.1 to about 1 wt. %, based on the total weight of the composition.

Thus, any one of the polyquaternium cationic polymer, if present, may be present, by weight, based on the total weight of the composition, from about 0.05, 0.06, 0.07, 0.08, 0.09, 0.1, 0.2, 0.3, 0.4, 0.5, 0.6, 0.7, 0.8, 0.9, 1, to about 2 weight percent, including increments and ranges therein and there between.

In the various embodiments, the composition may include other cationic polymers that are not a polyquaternium cationic polymer, each of which additional cationic polymer may be either nature-based or synthetic cationic polymer and may be present in the composition from about 0.05% to about 2%, or from about 0.05% to about 1%, or from about 0.05% to about 1%, or from about 0.1% to about 0.5%, or from about 0.1% to about 0.2% or any suitable combination, sub-combination, range, or sub-range thereof by weight, based on the weight of the cosmetic cleansing composition. One of ordinary skill in the art, however, will appreciate that other ranges are within the scope of the invention.

In accordance with the various embodiments, the amount of one or more additional ingredients, such as actives and additives, alone or in combination, when present in the composition according to the disclosure can be present in a range from about 0.0001% to about 20%, and in some embodiments, from about 0.005% to about 0.01%, and in some embodiments, from about 0.01% to about 0.1%, and in some embodiments, from about 0.15% to about 5%, and in some embodiments, from about 0.40% to about 4%, and in some embodiments, from about 0.5% to about 2.5%, and in some embodiments, from about 0.1% to about 0.5% and in some embodiments, from about 1% to about 2%, or any suitable combination, sub-combination, range, or sub-range thereof by weight, based on the total weight of the composition. One of ordinary skill in the art, however, will appreciate that other ranges are within the scope of the invention.

Thus, any one or a combination of additional ingredients, such as actives and additives may be present, each one or the combination present from about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 percent, by weight, including increments and ranges therein and there between.

Although the optional additives are given as examples, it will be appreciated that other optional components compatible with cosmetic applications known in the art may be used that are suitable. It will be appreciated by a skilled artisan that any optional additives are present only to the extent and in amounts that do not materially adversely affect the basic and novel characteristic(s) of the claimed disclosure. Thus, In some embodiments, that include optional additives, such optional additives will not materially adversely affect the foaming cleanser.

Further, any one or more (or all) of additional or optional additives and actives may be excluded from the composition. In other words, the composition may be free or essentially free of any or all of the additional or optional additives, actives, and other ingredients disclosed herein.

More generally, it will be appreciated by a skilled artisan that any solvents, humectants, preservatives or other additives are present only to the extent and in amounts that do not materially adversely affect the basic and novel characteristic(s) of the claimed invention.

### EXAMPLES

### Example 1: Compositions

Various representative embodiments of the inventive and comparative compositions are exemplified herein.

### Table 2: Inventive and Comparative Compositions

The table below provides compositional ingredients for each of compositions that include a Base (no thickener/Fatty alcohol—common to the inventive and comparative compositions), Comparative Composition (PEG thickener), and Inventive Compositions (including Fatty alcohol Hexyldecanol @0.1%, Fatty alcohol Hexyldecanol @0.3%, Fatty alcohol Oleyl @0.1%, Fatty alcohol Oleyl @0.3%, Fatty alcohol Lauryl/Mixed @0.1%, Fatty alcohol Lauryl/Mixed @0.3%), all amounts by weight, based on the weight of the composition.

| INCI | Base | Comp | INV 1 | INV 2 | INV 3 | INV 4 | INV 5 | INV 6 |
|---|---|---|---|---|---|---|---|---|
| COCAMIDOPROPYL | 8.25 | 8.25 | 8.25 | 8.25 | 8.25 | 8.25 | 8.25 | 8.25 |
| HYDROXYSULTAINE | | | | | | | | |
| SODIUM METHYL | 6.6 | 6.6 | 6.6 | 6.6 | 6.6 | 6.6 | 6.6 | 6.6 |
| COCOYL TAURATE | | | | | | | | |
| PEG-150 | | 0.3 | | | | | | |
| PENTAERYTHRITYL | | | | | | | | |
| TETRASTEARATE | | | | | | | | |
| HEXYLDECANOL | | | 0.1 | 0.3 | | | | |
| OLEYL ALCOHOL | | | | | 0.1 | 0.3 | | |
| LAURYL ALCOHOL | | | | | | | 0.1 | 0.3 |
| 69%, MYRISTYL | | | | | | | | |
| ALCOHOL 25%, Cetyl | | | | | | | | |
| alcohol 6% | | | | | | | | |
| WATER / AQUA | ~72/QS | ~72/QS | ~72/QS | ~72/QS | ~72/QS | ~72/QS | ~72/QS | ~72/QS |
| niacinamide, | ~13 | ~13 | ~13 | ~13 | ~13 | ~13 | ~13 | ~13 |
| phenoxyethanol, water / | | | | | | | | |
| aqua, sodium hyaluronate, | | | | | | | | |
| glycerin, sodium chloride, | | | | | | | | |
| sodium benzoate, sodium | | | | | | | | |
| lauroyl lactylate (and) | | | | | | | | |
| ceramide NP (and) | | | | | | | | |
| ceramide AP (and) | | | | | | | | |
| phytosphingosine (and) | | | | | | | | |
| cholesterol (and) xanthan | | | | | | | | |
| gum (and) carbomer (and) | | | | | | | | |
| ceramide EOP, | | | | | | | | |
| propanediol, tetrasodium | | | | | | | | |
| glutamate diacetate, citric | | | | | | | | |
| acid | | | | | | | | |
| sum composition | 100 | 100 | 100 | 100 | 100 | 100 | 100 | 100 |
| Viscosity, M3 (UD) | 7.3 | 25 | 16.6 | 38.5 | 12 | 21.3 | 12.4 | 27.8 |

* all formulas at pH 5.0

As shown above, the inventive compositions, particularly those having at least 0.3% fatty alcohol, perform at least comparatively, if not improved, relative to the PEG-Containing comparative composition sharing the same base with the inventive composition.

In stability tests, the inventive compositions demonstrate good to excellent overall stability, even after exposure to high heat for up to two months. Note, the Comparative comparison, which lacks the taurate anionic surfactant, demonstrates foaming volume and bubble size properties that are different from the inventive embodiments, with comparable stability.

### Example 1: Commercial Benchmark

TABLE 2
Commercial Benchmark Composition

| INCI US | Benchmark |
|---|---|
| COCAMIDOPROPYL HYDROXYSULTAINE | 6.48 |
| SODIUM METHYL COCOYL TAURATE | 1.00 |
| SODIUM LAUROYL SARCOSINATE | 4.47 |
| SODIUM LAUROYL LACTYLATE | 0.025 |
| PEG-150 PENTAERYTHRITYL TETRASTEARATE | 2.75 |
| PEG-6 CAPRYLIC/CAPRIC GLYCERIDES | 1.10 |
| WATER | ~75 |
| ceramide EOP, ceramide NP, ceramide AP, carbomer, | ~9.4 |
| xanthan gum, disodium EDTA, phytosphingosine, | |
| glycerin, propylene glycol, cholesterol, tetrasodium | |
| EDTA, sodium chloride, citric acid, hydrolyzed | |
| hyaluronic acid, niacinamide, propylparaben | |
| methylparaben | |
| Viscosity | 30 UD |

As shown in Tables 1 and 2, above, the inventive compositions, particularly those having at least 0.3% fatty alcohol, perform at least comparatively, if not improved relative to the benchmark composition which is thickened with PEG-thickeners in a generally similar base.

The results demonstrate that the inventive composition performs at least as well as the commercial benchmark and has more desirable foaming and textural/viscosity properties.

While the disclosure has been described with reference to an embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising,” “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

“Free” and “devoid” and “exclude” or “excluded” each indicates that no reliably measurable excluded material, for example, an excluded thickener or excluded sulfate surfactant or other excluded material as described herein, is present in the composition. The term “essentially free” means that, while it is preferred that no excluded material is present in the composition, it is possible to have very small amounts of the excluded material in the composition of the invention, provided that these amounts do not materially affect the advantageous properties of the composition. In particular, “essentially free” means that excluded material can be present in the composition at an amount of less than 5%, or less than 4%, or less than 3%, or less than 2%, or less than 1%, or less than 0.1% by weight, or at 0%, based on the total weight of the composition.

In accordance with the various embodiments, it is contemplated that some or all of following ingredients may be excluded from the composition according to the invention: alcohols, for example but not limited to, monohydric alcohols; powders comprising Nylon-12, polymethyl methacrylate, polyethylene beads, and powders which are considered microplastics; lactic acid, glycolic acid, tartaric acid, mandelic acid, citric acid, salicylic acid and derivatives thereof (including 5-n-octanoylsalicylic acid, salicylate, sodium salicylate, and willow extract), salicylic acid, capryloyl salicylic acid, beta hydroxybutanoic acid, propionic acid, beta-hydroxy beata-methylbutyric acid, carnitine tropic acid, and trethocanic acid; alkyl polyglucosides selected from the group consisting of decyl glucoside, lauryl glucoside, octyl glucoside, coco glucoside, caprylyl/capryl glucoside, and sodium lauryl glucose carboxylate, and combinations thereof; surfactants with a C10-C20 fatty alcohol or acid hydrophobe condensed with from about 2 to about 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan; mono- and di-C8-C20 fatty acids; polyoxyethylene sorbitan; alkyl polyglycosides and saccharide fatty amides (e.g. methyl gluconamides); alkyl ether sulfate and sulfonates; alkyl sulfates and sulfonates; alkylbenzene sulfonates; alkyl and dialkyl sulfosuccinates; C8-C20 acyl isethionates; C8-C20 alkyl ether phosphates; alkylethercarboxylate, PEG-100 Stearate; PEG-20 Stearate and other esters of Poly(Ethylene Glycol); polyethylene glycol (PEG) surfactants, polypropylene glycol (PPG) surfactants, sulfate surfactants; taurates, for example, taurate surfactants; Sucrose Stearate and other emulsifiers based on sugar esters; Glyceryl Stearate and other glycerol esters; Disodium Ethylene Dicocamide PEG-15 Disulfate; Sodium Steroyl Glutamate and other fatty amides; Steareth-100 and other fatty ethers, cationic surfactants, and other cationic compounds; silicones, such as silicone polymers, silicone elastomers, and silicone oils, for example selected from dimethicone, cyclopentasiloxane, cyclohexasiloxane, and combinations thereof; phthalates, parabens, sulfates, polyquaternium, polylysine, microplastics, synthetic dyes, gelling agents, and combinations thereof.

“Excluded polymeric thickeners” means and refers to thickeners that have not been added as a component in the composition or that may be excluded in some embodiments. Thus, in some embodiments, the composition is essentially free, free or devoid of one or more of PEG/PEO polymeric thickeners, nature based thickeners, and synthetic thickeners. In some embodiments, the composition is essentially free of or excludes PEG-type polymeric thickeners selected from PEG-150 pentaerythrityl tetrastearate, PEG-120 thickeners, PEG-150 thickeners, PEG-80 glyceryl tallow ester, PEG-8 PPG (polypropylene glycol)-3 diisostearate, PEG-200 hydrogenated glyceryl palmitate, PEG-n (n=6, 8, 12) beeswax, PEG-4 isostearate, PEG-n (n=3, 4, 8, 150) distearate PEG-n (n=28, 200) glyceryl tallow ester, PEG-7 hydrogenated castor oil, PEG-40 jojoba oil, PEG-120 methyl glucose dioleate, PEG-150 pentaerythritol stearate, PEG-55 propylene glycol oleate, PEG-160 sorbitan triisostearate, SMDI copolymer (polyethylene glycol-150/decyl/methacrylate copolymer), PEG-150/stearyl/SMDI copolymer, PEG-90, and combinations thereof. In some embodiments, the composition may exclude one or more polymers such as cationic polymers selected from polylysine, chitosan, and guar. In some embodiments, the composition may exclude one or more polymers selected from polysaccharides that include alginates/algin, chitosan, cyclodextrin, cationic gelatin, cationic dextran, celluloses, cationic cellulose, chitin, starches, galactomannans such as guar gums and its derivatives, including hydroxypropyl guar, cationic guar derivatives, gums of microbial origin, including xanthan gum, scleroglucan gum, mucopolysaccharides, chondroitin sulfates, and mixtures thereof, and gums derived from plant exudates, including locust bean gums, gum arabic, ghatti gum, karaya gum, gum tragacanth, carrageenan gum, agar gum and carob gum, cellulose-based polymers, including methylcellulose, hydroxyalkylcellulose, ethylhydroxyethylcelluloses, hydroxyethylcellulose, hydroxypropylcellulose, carboxymethylcellulose, cationic cellulose ether derivatives, quaternized cellulose derivatives, nitrocellulose, hemicellulose and hemicellulose derivatives, mannans, xylans, lignins, arabans, galacturonans, alginate-based compounds, glucuronoxylans, arabinoxylans, xyloglucans, glucomannans, fructosans such as inulin, pectic acids and pectins, arabinogalactans, agars, glycosaminoglycans, gelatin, gellan, crosslinked homopolymers of acrylic acid, cationic peptides and their derivatives (e.g., polylysine, polyornithine), peptide/protein polymers (e.g., histone, collagen); cationic thiolated biopolymers (nature-based thiomers or nature-based dendrimers, e.g., chitosan-cysteine, chitosan-thiobutylamidine, chitosan-thioglycolic acid); algin, xanthan gum (and) sclerotium gum (and) lecithin (and) pullulan (Siligel™), xanthan gum, carbomer 980, hydroxypropyl starch phosphate, ammonium polyacryloyldimethyl taurate, hydroxyethyl cellulose, acrylate copolymer, sclerotium gum; or combinations thereof. In some embodiments, the composition may also exclude one or more PEG type compound selected from poloxamer 184 (polyoxyethylene polyoxypropylene glycol), PEG-20 glyceryl triisostearate, PEG-7 glyceryl cocoate, PEG-20 methylglucoside sesquistearate, PG-5 dioleate, PG-4 diisostearate, PG-10 isostearate, PEG-60 hydrogenated castor oil, PEG-8 stearate, PEG-8 isostearate, PEG-60 hydrogenated castor oil, PEG-150 Laurate, PEG-150 Distearate, PEG-150 pentaerythrityl tetrastearate, PEG-78 Glyceryl Cocoate, PEG-30 Glyceryl Cocoate, PEG-type polymeric thickeners selected from PEG-150 pentaerythrityl tetrastearate, PEG-120 thickeners, PEG-150 thickeners, PEG-80 glyceryl tallow ester, PEG-8 PPG (polypropylene glycol)-3 diisostearate, PEG-200 hydrogenated glyceryl palmitate, PEG-n (n=6, 8, 12) beeswax, PEG-4 isostearate, PEG-n (n=3, 4, 8, 150) distearate PEG-n (n=28, 200) glyceryl tallow ester, PEG-7 hydrogenated castor oil, PEG-40 jojoba oil, PEG-120 methyl glucose dioleate, PEG-150 pentaerythritol stearate, PEG-55 propylene glycol oleate, PEG-160 sorbitan triisostearate, SMDI copolymer (polyethylene glycol-150/decyl/methacrylate copolymer), PEG-150/stearyl/SMDI copolymer, PEG-90, -20, Ceteth-10, Ceteth-20, Isoceteth-20, Laureth-4, Laureth-23, Oleth-10, Oleth-20, Steareth-10, Steareth-20, Steareth-100, and Steareth-21, PEG-20 glyceryl laurate, PEG-40 hydrogenated castor oil, or combinations thereof.

“Sulfate surfactant-free” means that excluded surfactants have not been added as a component. In some particular embodiments, the composition is devoid of sulfate based surfactant. In some embodiments, the composition is devoid of ethylene glycol (PEG) based surfactants. Those of skill in the art will appreciate that a surfactant may be present in a composition via its presence in one or more of the formulation components; thus, In some embodiments, the composition may be “essentially-free” wherein the excluded surfactant is present at a concentration that does not exceed 5% by weight, and in some instances is present not more than 3% by weight, and in some instances is present not more than 1% by weight, based on the weight of the cosmetic composition. In some particular embodiments “sulfate surfactant-free” means that the cosmetic composition is free or devoid specifically of the excluded sulfate surfactant.

“Petrochemical-free” means that an excluded petrochemical has not been added as a component. Some specific but non-limiting examples of petrochemicals that are lacking from the cosmetic composition include benzalkonium chloride, isododecane, isohexadecane and the like. In some embodiments, the composition is not free from petrochemicals, but is formulated to have an amount of petrochemicals that is at or below the amounts found in similar compositions that are commercially available. The composition may be petrochemical-free.

“Silicone-free” means that an excluded silicones has not been added as a component. In some embodiments, a composition is devoid of silicones. Some specific but non-limiting examples of silicones that are lacking from the cosmetic composition includes, but is not limited to, silicone polymers, for example selected from dimethicone, cyclopentasiloxane, and other silicone oils, and silicone elastomers. The composition may be silicone-free.

Other than in the operating examples, or where otherwise indicated, all numbers expressing quantities of ingredients and/or reaction conditions may be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “²% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

What is claimed is:

1. A foaming cleanser comprising: (a) at least one fatty alcohol; (b) at least one amphoteric surfactant; (c) at least one anionic surfactant; and (d) water; wherein the foaming cleanser excludes PEG based ingredients.

2. The foaming cleanser according to claim 1, wherein the foaming cleanser excludes PEG/PEO polymeric thickeners, parabens, and combinations thereof.

3. The foaming cleanser according to claim 1, wherein the foaming cleanser excludes PEG-150 pentaerythrityl tetrastearate, PEG-6 caprylic/capric glycerides, or a combination thereof.

4. The foaming cleanser according to claim 1, wherein the at least one fatty alcohol is present from about 0.05% to about 2% by weight, based on the weight of the composition.

5. The foaming cleanser according to claim 1, wherein the at least one fatty alcohol is present from about 0.05% to about 1% by weight, based on the weight of the composition.

6. The foaming cleanser according to claim 1, wherein the fatty alcohol is present in a total amount that does not exceed 2% by weight, based on the weight of the composition.

7. The foaming cleanser according to claim 1, wherein the at least one fatty alcohol is characterized by a feature selected from the group consisting of: (i) being liquid or semi-waxy at room temperature and containing carbon chain length from 6 up to 22; (ii) being liquid or semi-waxy at room temperature and containing carbon chain length from 6 up to 18; (iii) being liquid or semi-waxy at room temperature and containing carbon chain length from 8 up to 10; (iv) being liquid or semi-waxy at room temperature and containing carbon chain length from 10 up to 16; (v) being selected from linear or branched, saturated or unsaturated, with hydroxyl group on terminal position or non-terminal position; (vi) being selected from the group consisting of hexyldecanol, oleoyl alcohol, lauryl alcohol, myristyl alcohol, cetyl alcohol, decanol, butyloctanol, hexydecanol, palmitoleyl alcohol, and combinations thereof; and (vii) combinations thereof.

8. The foaming cleanser according to claim 1, wherein the at least one amphoteric surfactant is characterized by a feature selected from the group consisting of: (i) present from about 1% to about 15% by weight, based on the weight of the composition; (ii) present from about 3% to about 10% by weight, based on the weight of the composition; (iii) selected from the group consisting of cocoamidopropyl hydroxysultaine, coco betaine, cocoamidopropyl betaine, and combinations thereof; and (iv) combinations thereof.

9. The foaming cleanser according to claim 1, wherein the at least one anionic surfactant is selected from the group consisting of sarcosinate, glycinate, glutamate, taurate, isethionate, alpha olefin sulfonate, and combinations thereof.

10. The foaming cleanser according to claim 1, further comprising an additional ingredient selected from the group consisting of niacinamide, phenoxyethanol, sodium hyaluronate, glycerin, sodium chloride, sodium benzoate, sodium lauroyl lactylate (and) ceramide NP (and) ceramide AP (and) phytosphingosine (and) cholesterol (and) xanthan gum (and) carbomer (and) ceramide EOP, propanediol, tetrasodium glutamate diacetate, citric acid, and combinations thereof.

11. A foaming cleanser comprising: (a) at least one fatty alcohol present from about 0.05% to about 2% and selected from the group consisting of decanol, butyloctanol, lauryl alcohol, myristyl alcohol, hexydecanol, palmitoleyl alcohol, oleoyl alcohol, and combinations thereof; (b) at least one amphoteric surfactant present from about 1% to about 15% and selected from the group consisting of cocoamidopropyl hydroxysultaine, coco betaine, cocoamidopropyl betaine, and combinations thereof; (c) at least one anionic surfactant present from about 1% to about 15% and selected from the group consisting of sarcosinate, glycinate, glutamate, taurate, isethionate, alpha olefin sulfonate, and combinations thereof; all amounts by weight, based on the weight of the composition.

12. The foaming cleanser according to claim 11, wherein the foaming cleanser excludes PEG/PEO polymeric thickeners, parabens, and combinations thereof.

13. The foaming cleanser according to claim 11, wherein the at least one fatty alcohol consists of (i) hexyldecanol, (ii) oleoyl alcohol, or (iii) a blend of lauryl alcohol, myristyl alcohol, and cetyl alcohol.

14. The foaming cleanser according to claim 11, wherein the at least one amphoteric surfactant is present from about 3% to about 10% by weight, based on the weight of the composition, and the at least one anionic surfactant is present from about 3% to about 10% by weight, based on the weight of the composition.

15. The foaming cleanser according to claim 11, wherein each of the at least one fatty alcohol is liquid at room temperature and contains a carbon chain length from 8 up to 10, and the at least one amphoteric surfactant contains a carbon chain length distribution from C8-10, or each of the at least one fatty alcohol is liquid at room temperature and contains a carbon chain length from 16 up to 18, and the at least one amphoteric surfactant contains a carbon chain length distribution from C16-18.

16. The foaming cleanser according to claim 11, further comprising an additional ingredient selected from the group consisting of niacinamide, phenoxyethanol, water/aqua, sodium hyaluronate, glycerin, sodium chloride, sodium benzoate, sodium lauroyl lactylate (and) ceramide NP (and) ceramide AP (and) phytosphingosine (and) cholesterol (and) xanthan gum (and) carbomer (and) ceramide EOP, propanediol, tetrasodium glutamate diacetate, citric acid, and combinations thereof.

17. A foaming cleanser comprising: (a) at least one fatty alcohol present from about 0.05% to about 2% and selected from the group consisting of (i) hexyldecanol, (ii) oleoyl alcohol, (iii) a blend of lauryl alcohol, myristyl alcohol, and cetyl alcohol, (iv) and combinations thereof, wherein the fatty alcohol is present in a total amount that does not exceed 2% by weight, based on the weight of the composition; (b) at least one amphoteric surfactant present from about 3% to about 10% and comprising at least cocoamidopropyl hydroxysultaine; (c) at least one anionic surfactant present from about 3% to about 10% and comprising at least sodium methyl cocoyl taurate; (d) an additional ingredient selected from the group consisting of niacinamide, phenoxyethanol, water/aqua, sodium hyaluronate, glycerin, sodium chloride, sodium benzoate, sodium lauroyl lactylate (and) ceramide NP (and) ceramide AP (and) phytosphingosine (and) cholesterol (and) xanthan gum (and) carbomer (and) ceramide EOP, propanediol, tetrasodium glutamate diacetate, citric acid, and combinations thereof; all amounts by weight, based on the weight of the composition; wherein the foaming cleanser excludes PEG/PEO polymeric thickeners, parabens, and combinations thereof.
